(19) United States
(12) Patent Application Publication
(10) Pub. No.: US 2025/0351858 A1
(43) Pub. Date: Nov. 20, 2025

(54) PLANT-DERIVED COMPOSITION

(71) Applicant: NANJING NUTRABUILDING BIO-TECH CO., LTD., Nanjing (CN)

(72) Inventors: Jun TANG, Nanjing (CN); Shijie XU, Nanjing (CN); Wei WANG, Nanjing (CN); Kylin LIAO, Nanjing (CN); Lang LANG, Nanjing (CN)

(21) Appl. No.: 19/232,312

(22) Filed: Jun. 9, 2025

Related U.S. Application Data
(63) Continuation of application No. PCT/CN2023/136299, filed on Dec. 5, 2023.

(30) Foreign Application Priority Data
Dec. 9, 2022 (CN) 202211580569.0

(51) Int. Cl. A23L 33/105; A61K 9/16; A61K 31/05; A61K 31/352; A61K 36/31; A61K 36/45; A61K 36/53; A61K 36/534; A61K 36/54; A61K 36/63; A61K 36/73; A61K 36/82; A61K 36/8962; A61K 36/9066

(52) U.S. Cl. CPC A23L 33/105; A61K 9/1611; A61K 9/1623; A61K 9/1652; A61K 31/05; A61K 31/352; A61K 36/31; A61K 36/45; A61K 36/53; A61K 36/534; A61K 36/54; A61K 36/63; A61K 36/73; A61K 36/82; A61K 36/8962; A61K 36/9066

(57) ABSTRACT

The invention discloses a plant-derived composition, comprising, by weight, 30%-50% of a first plant component, 37%-57% of a second plant component, and 5%-20% of a third plant component. The plant-derived composition of the invention has high antioxidant capacity, can efficiently scavenge human free radicals, and can be used as a composite dietary supplement for supplementing essential nutrients for human body.

### CROSS REFERENCE TO RELATED APPLICATION

This application is a continuation application of International Patent Application No. PCT/CN2023/136299, filed on Dec. 5, 2023, which claims the priority of the Chinese patent application CN2022115805690, filed on Dec. 9, 2022, the contents of all of which are incorporated herein by reference in their entirety.

### FIELD OF THE INVENTION

The invention belongs to the technical field of health foods and dietary supplements. In particular, the invention relates to a plant-derived composition with high antioxidant capacity.

### BACKGROUND OF THE INVENTION

Free Radical is the intermediate metabolite of various biochemical reactions in human life activities, which has high chemical activity and is an effective defense system of the body. If it cannot be maintained at a certain level, it will affect the life activities of the body. In the process of human metabolism, various kinds of free radicals are constantly produced, including various free radicals will be produced continuously, including Oxygen Radicals, Hydroxyl Radicals, Superoxide anion [O₂⁻] Radicals, peroxynitrite [ONOO⁻] Radicals and Singlet Oxygen. When these free radicals are excessive in the body, a series of biological reactions will be triggered, leading to physical decline and accompanied by pathological changes. Antioxidants can efficiently scavenge free radicals and prevent or inhibit various disorders or diseases caused by excessive free radicals.

Due to the structural limitation of single-component antioxidant, it can't completely scavenge free radicals with different properties, and its antioxidant activity is low when used alone, and it is easy to produce toxic and side effects when used in large amounts. Through reasonable formula and proportion, various antioxidant components can produce synergistic effect, and the complementary effect of their structure-activity relationship can play a stronger role than a single antioxidant. Composite antioxidants can be regenerated by mutual repair, forming a redox circulation system, so that the antioxidant activity is significantly stronger than that of single antioxidants. However, the antioxidant activity of different structural types is different, and even the antioxidant activity of the same structural type will be affected by the number and position of substituents. The antioxidant synergistic effect or antagonistic effect between antioxidant components is influenced by the structure, concentration and reaction system of antioxidants. Improper proportion will lead to antagonism, and there is a strong correlation between antagonism and its proportion. Therefore, it is of great significance to study the synergistic effect between antioxidants and find a composite natural antioxidant with high efficiency and low toxicity for the efficient use of antioxidants.

At present, the existing antioxidant single component or composition cannot reach a high level of Oxygen Radical Absorbance Capacity for the time being. So, it is urgent to develop a product of plant-derived/plant-based composition with high antioxidant ability to effectively scavenge human free radicals and prevent or inhibit various disorders, diseases, or problems caused by excessive free radicals in the human body, and this kind of product will have a very broad market prospect.

### SUMMARY OF THE INVENTION

Aiming at the shortcomings of the prior art, the invention provides a product of a plant-derived composition with high antioxidant capacity to efficiently scavenge human free radicals, and can be used as a composite dietary supplement to supplement essential nutrients for human body, thus achieving an ideal health care effect.

In one aspect, the invention provides a plant-derived composition comprises a first plant component, a second plant component and a third plant component, wherein the first plant component is selected from one or more of quercetin, green tea extract, broccoli bud extract, onion extract, apple extract, tomato extract, broccoli extract, Camu fruit extract, Acerola cherry extract, blueberry extract, Prunus avium extract, raspberry extract, spinach extract, aronia melanocarpa extract, kale extract, blackberry extract, cowberry extract; the second plant component is selected from one or more of olive extract, clove extract, resveratrol; the third plant component is selected from one or more of Rosmarinus officinalis extract, grape seed extract.

In some embodiments, the plant-derived composition comprises, by weight, 30%-50% of the first plant component, 37%-57% of the second plant component and 5%-20% of the third plant component. Preferably, the amount of the first component is 33%-49%, 35%-47%, 38%-45% by weight; the amount of the second component is 40%-56%, 43%-53%, 45%-50% by weight; and the amount of the third component is 7%-18%, 9%-16%, 11%-14% by weight.

In some embodiments, the plant-derived composition further comprises 0.5%-2% by weight of a fourth plant component, and the fourth plant component is selected from one or more of turmeric extract, garlic extract, basil extract, oregano extract, cinnamon extract, carrot extract, Sambucus williamsii extract, mangosteen extract.

In some embodiments, the plant-derived composition further comprises 0.5%-2% by weight of a fifth plant component, and the fifth plant component is selected from one or more of basil extract, oregano extract, cinnamon extract, Sambucus williamsii extract.

In some embodiments, the plant-derived composition further comprises a filler, a glidant, or an additive.

In some embodiments, the filler includes one or more of resistant dextrin, maltodextrin, lactose; the glidant includes one or more of silicon dioxide, magnesium stearate, talcum powder, tricalcium phosphate; the additive includes one or more of ergothioneine, L-ascorbic acid, sodium D-erythorbate, tea polyphenol.

In some embodiments, the first plant component is quercetin, kale extract; the second plant component is olive extract; the third plant component is Rosmarinus officinalis extract.

In some embodiments, the first plant component is quercetin, kale extract; the second plant component is olive extract; the third plant component is Rosmarinus officinalis extract; the fourth plant component is turmeric extract; the fifth plant component is cinnamon extract.

In some embodiments, the above composition further comprises resistant dextrin, silicon dioxide, ergothioneine.

In another aspect, the invention provides a method for using the plant-derived composition as described above, wherein the method for using comprises administering the plant-derived composition to a subject in need thereof in an amount of 0.1 g-200 g per day.

In some embodiments, the plant-derived composition can be administered 0.1-150 g, 0.2-100 g, 0.5-50 g, 1-20 g, 2-15 g, 4-10 g, 6-9 g per day. In some embodiments, the plant-derived composition can be administered once or more times a day.

In some embodiments, the plant-derived composition is prepared as a solid preparation or a liquid preparation. In some embodiments, the plant-derived composition is prepared in powder form. In some embodiments, the plant-derived composition can be administered alone, or together with or mixed with other nutritional supplements, foods or beverages. In some embodiments, the plant-derived composition can be mixed into yogurt or bread flour in powder form.

In some embodiments, the invention provides use of the plant-derived composition of the invention for preparing foods, beverages, nutraceuticals, dietary supplements, cosmetics.

The plant-derived composition of the invention is beneficial to the balanced collocation of nutrients needed by human body. It mainly comes from fruits, vegetables and other plants, which are easy to obtain, green in source, safe in ingredients and easily absorbed by the intestines. It tastes good and improves the adaptability of users. Moreover, it achieves a high antioxidant capacity that cannot be achieved by a single plant component or the components of the existing composite composition on the market, and can efficiently scavenge human free radicals and prevent or inhibit various disorders, diseases or problems caused by excessive free radicals in the human body.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” or their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

As used herein, the terms “subject” or “individual” are used interchangeably to refer to any subject to whom the disclosed methods and compositions can be applied or administered. The subject may have a disease or disorder, but the subject does not need to be sick to benefit from the disclosed methods and compositions. Therefore, any subject can take the disclosed composition or become a recipient of the disclosed method. As used herein, the term “subject” refers to animals (for example, birds, reptiles and mammals). In some embodiments, the subject can be mammals including non-primates (e.g., camels, donkeys, zebras, cows, horses, cats, dogs, rats and mice) and primates (e.g., monkeys, chimpanzees and humans). In certain embodiments, the subject may be a non-human mammal. In other embodiments, the subject may be a human.

As used herein, the term “administration” refers to the process of delivering a disclosed composition or active ingredient to a subject. The compositions of the invention can be administered in a variety of ways, including orally, intragastrically, and parenterally (e.g., intravenous and intraarterial as well as other suitable parenteral routes), and the like, so as to exert the desired effects.

As used herein, the term “plant component” is any material based on or derived from any kind of plant substances, which means a material containing the whole plant itself, a part of the plant itself (such as leaves, roots, skins, flesh, pulp, etc.) or its processed products, which may include, for example, plant extracts, plant concentrates, dried plants, plant pulp, plant powder, plant fibers, and the like. In some embodiments, plant components can be based on fruits, vegetables, common traditional Chinese medicines with Medicine and Food Homology, etc.

The plant-derived composition of the invention is simple and convenient to prepare, and can be prepared by uniformly mixing all the raw materials. In a preferred embodiment, the preparation method of the plant-derived composition of the invention comprises the following steps: firstly, uniformly mixing the raw materials with low content in the raw materials, and then mixing them with the raw materials with high content in equal increments. More preferably, the raw materials are mixed in a three-dimensional way for 20-30 minutes. In the preparation method of the invention, functional components can be reasonably added in the above steps depending on the properties of raw materials.

The technical features, implementation methods and beneficial effects of the invention will be further described in detail with specific examples. The following examples are only part of the embodiments of the present invention, but not all of them. Unless otherwise specified, the materials and reagents described in the following examples are all common commercial products and can be purchased in the market.

### EXAMPLES

These examples describe exemplary plant-derived compositions.

### Example 1 Screening of Plant Extracts with High Antioxidant Capacity

Determination of ORAC value and screening: Pretreatment of sample solution: About 20 mg of plant extract was weighed, put in a 50 mL volumetric flask. 30 mL of 70% ethanol was added and ultrasonically treated for 40 minutes, and the volume was adjusted to scale with 70% ethanol. It was transferred to a centrifuge tube, centrifuged at 4000R for 20 minutes. The supernatant was collected and diluted continuously (diluent: PBS) to a reasonable linear range. PBS preparation: 8 g sodium chloride, 0.2 g KCl, 0.27 g potassium dihydrogen phosphate, 3.58 g disodium hydrogen phosphate and 39 mg DTPA was weighed, put in 1 L solution and stored at room temperature. Preparation of Trolox linear solution (mother liquor concentration 5 mM): 10 μL of 5 mM mother liquor was added to 240 μL of PBS to prepare 0.2 mM solution.

| Standard | 0.2 mM Trolox | Assay diluent | Concentration |
|---|---|---|---|
| linear solution | (μL) | (μL) | (μM) |
| 1 | 50 | 150 | 50 |
| 2 | 40 | 160 | 40 |
| 3 | 30 | 170 | 30 |
| 4 | 20 | 180 | 20 |
| 5 | 10 | 190 | 10 |
| 6 | 5 | 195 | 5 |
| 7 | 2.5 | 197.5 | 2.5 |
| 8 | 0 | 200 | 0 |

Operations following the instructions of the kit: Turn on the microplate reader and set the incubation temperature at 37° C. Dilute 4× diluent with deionized water to 1× assay diluent. Dilute the 100× fluorescent probe with 1× diluent into 1× fluorescent probe. Add 25 μL of PBS, sample solution and control solution respectively to a 96-well plate. Add 150 μL of 1× fluorescent probe into a 96-well plate. Transfer the 96-well plate to microplate reader at 37° C. in the dark, and incubate for 30 minutes. Set the parameters of the microplate reader (run for 60 min, and read a value every 2 min; EX/EM=480/520 nm; Strength: medium to high, sample placement area, shaking). Prepare 80 mg/mL initiator with PBS. Immediately take 25 μL of the prepared initiator and add it into the 96-well plate. Immediately put it into the microplate reader to run.

Various kinds of plant extracts were screened by Oxygen Radical Absorbance Capacity (ORAC), and it was found that quercetin, green tea extract, broccoli bud extract, onion extract, apple extract, tomato extract, broccoli extract, Camu fruit extract, Acerola cherry extract, blueberry extract, Prunus avium extract, raspberry extract, spinach extract, Aronia melanocarpa extract, kale extract, blackberry extract, cowberry extract, etc. or composition comprising the same have higher ORAC value, preferably up to 20,000 μmole TE/g.

Determination of HORAC value and screening: Pretreatment of sample solution: About 20 mg of plant extract was weighed, put in a 50 mL volumetric flask. 30 mL of 70% ethanol was added and ultrasonically treated for 40 minutes, and the volume was adjusted to scale with 70% ethanol. It was transferred to a centrifuge tube, centrifuged at 4000R for 20 minutes. The supernatant was collected and diluted continuously (diluent: PBS) to a reasonable linear range. PBS preparation: 8 g sodium chloride, 0.2 g KCl, 0.27 g potassium dihydrogen phosphate, 3.58 g disodium hydrogen phosphate and 39 mg DTPA was weighed, put in 1 L solution and stored at room temperature. Preparation of Gallic acid linear solution (mother liquor concentration 5 mM): 10 μL of 5 mM mother liquor was added to 240 μL of PBS to prepare 0.2 mM solution.

| Standard linear | Gallic acid | Assay diluent | Concentration |
|---|---|---|---|
| solution | (μL) | (μL) | (μM) |
| 1 | 90 | 410 | 900 |
| 2 | 80 | 420 | 800 |
| 3 | 70 | 430 | 700 |
| 4 | 60 | 440 | 600 |
| 5 | 50 | 450 | 500 |
| 6 | 40 | 460 | 400 |
| 7 | 30 | 470 | 300 |
| 8 | 20 | 480 | 200 |
| 9 | 10 | 490 | 100 |
| 10 | 0 | 500 | 0 |

Operations following the instructions of the kit: Turn on the microplate reader and set the incubation temperature at 37° C. Dilute 4× diluent with deionized water to 1× assay diluent. Dilute the 100× fluorescent probe with 1× diluent into 1× fluorescent probe. Add 25 μL of PBS, sample solution and control solution respectively to a 96-well plate. Add 140 μL of 1× fluorescent probe into a 96-well plate. Transfer the 96-well plate to microplate reader at 37° C. in the dark, and incubate for 30 minutes. Set the parameters of the microplate reader (run for 60 min, and read a value every 2 min; EX/EM=480/520 nm; Strength: automatic, sample placement area, shaking). Dilute 5× initiator with deionized water to a 1× solution (hydroxyl radical initiator). Add 20 μL of 1× initiator solution into 96-well plate. Add 20 μL of Fenton reagent solution into 96-well plate. Immediately put it into the microplate reader to run.

Various kinds of plant extracts were screened by Hydroxyl Radical Absorbance Capacity (HORAC), and it was found that olive extract, olive extract, resveratrol, etc. or combination thereof have higher HORAC value, preferably up to 11,000 μmole TE/g.

Determination of SORAC value and screening: Pretreatment of sample solution: About 20 mg of plant extract was weighed, put in a 50 mL volumetric flask. 30 mL of 70% ethanol was added and ultrasonically treated for 40 minutes, and the volume was adjusted to scale with 70% ethanol. It was transferred to a centrifuge tube, centrifuged at 4000R for 20 minutes. The supernatant was collected and diluted continuously (diluent: PBS) to a reasonable linear range. PBS preparation: 8 g sodium chloride, 0.2 g KCl, 0.27 g potassium dihydrogen phosphate, 3.58 g disodium hydrogen phosphate and 39 mg DTPA was weighed, put in 1 L solution and stored at room temperature. Preparation of Trolox linear solution (mother liquor concentration 2 mM, molecular weight=250.29): Preparation of Trolox solution with a concentration of 2 mM: 5 mg of Trolox was weighed and added to 1 mL of DMSO to dissolve (20 mM), 0.5 mL of which was added to 4.5 mL PBS.

| Standard linear | | | Concentration |
|---|---|---|---|
| solution | Trolox (μL) | PBS (μL) | (μM) |
| 1 | 500 | 500 | 1000 |
| 2 | 200 | 800 | 400 |
| 3 | 100 | 900 | 200 |
| 4 | 50 | 950 | 100 |
| 5 | 25 | 975 | 50 |
| 6 | Add 4, 90 μL | 210 | 30 |
| 7 | Add 4, 80 μL | 320 | 20 |
| 8 | Add 4, 40 μL | 360 | 10 |
| 9 | Add 4, 20 μL | 380 | 5 |
| 10 | 0 | 4500 | 0 |

Turn on the microplate reader and set the incubation temperature at 37° C. Xanthine solution: weigh 15 mg of xanthine, put it in a 100 mL volumetric flask, add 5 mL of 0.1 N sodium hydroxide, and after ultrasonic dissolution, add PBS buffer solution to dilute the volume to the scale. Mother liquor of dihydroethidine fluorescent probe: weigh 5 mg of dihydroethidine, add 2 mL acetonitrile to dilute and dissolve, and store at −20° C. Dihydroethidine fluorescence working solution: accurately measure 20 μL of mother liquor of dihydroethidine fluorescence probe, and add 2 mL of xanthine solution. Add 25 μL of PBS, sample solution and control solution respectively to a 96-well plate. Add 150 μL of dihydroethidine fluorescent probe working solution into 96-well plate. Transfer the 96-well plate to microplate reader at 37° C. in the dark, and incubate for 20 minutes. Set the parameters of the microplate reader (run for 40 min, and read a value every 2 min; EX/EM=470/584 nm; Strength: medium to high, sample placement area, shaking). Xanthine oxidase solution: weigh 3.5 mg of xanthine oxidase (SIGMA0.08 u/mg), add 0.5 mL of PBS to dissolve, and mix evenly. Add 25 μL of xanthine oxidase solution into 96-well plate. Immediately put it into the microplate reader to run.

Various kinds of plant extracts were screened by Superoxide anion [02-] Radical Absorbance Capacity (SORAC), and it was found that olive extract, Rosmarinus officinalis extract, grape seed extract, etc. or combination thereof have higher SORAC value, preferably up to 5,000 μmole TE/g.

Determination of NORAC value and screening: Pretreatment of sample solution: About 20 mg of plant extract was weighed, put in a 50 mL volumetric flask. 30 mL of 70% ethanol was added and ultrasonically treated for 40 minutes, and the volume was adjusted to scale with 70% ethanol. It was transferred to a centrifuge tube, centrifuged at 4000R for 20 minutes. The supernatant was collected and diluted continuously (diluent: PBS) to a reasonable linear range. PBS preparation: 8 g sodium chloride, 0.2 g KCl, 0.27 g potassium dihydrogen phosphate, 3.58 g disodium hydrogen phosphate and 39 mg DTPA was weighed, put in 1 L solution and stored at room temperature. Preparation of Trolox linear solution (mother liquor concentration 2 mM, molecular weight=250.29): Preparation of Trolox solution with a concentration of 2 mM: 5 mg of Trolox was weighed and added to 1 mL of DMSO to dissolve (20 mM), 0.5 mL of which was added to 4.5 mL PBS.

| Standard linear | Trolox | PBS | Concentration |
|---|---|---|---|
| solution | (μL) | (μL) | (μM) |
| 1 | 100 | 900 | 200 |
| 2 | 50 | 950 | 100 |
| 3 | 25 | 975 | 50 |
| 4 | Add 2, 90 μL | 210 | 30 |
| 5 | Add 2, 80 μL | 320 | 20 |
| 6 | Add 5, 100 μL | 100 | 10 |
| 7 | Add 6, 100 μL | 100 | 5 |
| 8 | Add 5, 20 μL | 180 | 2 |
| 9 | 0 | 4500 | 0 |

Turn on the microplate reader and set the incubation temperature at 37° C. Preparation of DHR-123 fluorescent mother liquor (5 mM): Accurately weigh about 3.46 mg of DHR-123, add 2 mL of N, N-dimethylformamide and store at −80° C. DHR-123 fluorescent working solution (12.5 μM): accurately measure 10 μL of fluorescent mother liquor, add 4 mL PBS and mix well. Mother liquor of SIN-1 initiator: accurately weigh 2 mg of SIN-1, add 5 mL of N, N-dimethylformamide and store at −80° C. Working solution of SIN-1 initiator: accurately measure 40 μL, add 2 mL PBS and mix well. Add 25 μL of PBS, sample solution and control solution respectively to a 96-well plate. Set the parameters of the microplate reader (run for 30 min, and read a value every 2 min; EX/EM=470/525 nm; Strength: automatic, sample placement area, shaking). Add 150 μL of DHR-123 fluorescent probe working solution into 96-well plate. Add 25 μL of working solution of SIN-1 initiator into 96-well plate. Immediately put it into the microplate reader to run.

Various kinds of plant extracts were screened by peroxynitrite [ONOO⁻] Radical Absorbance Capacity (NORAC), and it was found that turmeric extract, garlic extract, basil extract, oregano extract, cinnamon extract, carrot extract, Sambucus williamsii extract, mangosteen extract, etc. or combination thereof have higher NORAC value, preferably up to 1400 μmole TE/g.

Determination of SOAC value and screening: Pretreatment of sample solution: About 20 mg of plant extract was weighed, put in a 50 mL volumetric flask. 30 mL of 70% ethanol was added and ultrasonically treated for 40 minutes, and the volume was adjusted to scale with 70% ethanol. It was transferred to a centrifuge tube, centrifuged at 4000R for 20 minutes. The supernatant was collected and diluted continuously (diluent: PBS) to a reasonable linear range. PBS preparation: 8 g sodium chloride, 0.2 g KCl, 0.27 g potassium dihydrogen phosphate, 3.58 g disodium hydrogen phosphate and 39 mg DTPA was weighed, put in 1 L solution and stored at room temperature. Preparation of Trolox linear solution (mother liquor concentration 2 mM, molecular weight=250.29): Preparation of Trolox solution with a concentration of 2 mM: 5 mg of Trolox was weighed and added to 1 mL of DMSO to dissolve (20 mM), 0.5 mL of which was added to 4.5 mL PBS.

| Standard linear | Trolox | PBS | Concentration |
|---|---|---|---|
| solution | (μL) | (μL) | (μM) |
| 1 | 500 | 500 | 1000 |
| 2 | 200 | 800 | 400 |
| 3 | 100 | 900 | 200 |
| 4 | 50 | 950 | 100 |
| 5 | 25 | 975 | 50 |
| 6 | Add 4, 90 μL | 210 | 30 |
| 7 | Add 4, 80 μL | 320 | 20 |
| 8 | Add 4, 40 μL | 360 | 10 |
| 9 | Add 4, 20 μL | 380 | 5 |
| 10 | 0 | 4500 | 0 |

Turn on the microplate reader and set the incubation temperature at 37° C. Mother liquor of dihydroethidine fluorescent probe: weigh 5 mg of dihydroethidine, add 2 mL acetonitrile to dilute and dissolve, and store at −20° C. Dihydroethidine fluorescence working solution: accurately measure 150 μL of mother liquor of dihydroethidine fluorescence probe, and add 2 mL of PBS and mix well. Preparation of hydrogen peroxide solution: accurately measure 1.5 mL of 30% hydrogen peroxide, put it in a 50 mL volumetric flask, and add PBS to dilute the volume to the scale. Preparation of sodium molybdate solution: accurately weigh 40 mg of sodium molybdate, put it in a 50 mL volumetric flask, add PBS for ultrasonic dissolution, and dilute the volume to the scale. Add 25 μL of PBS, sample solution and control solution respectively to a 96-well plate. Add 125 μL of fluorescent probe working solution into 96-well plate. Transfer the 96-well plate to microplate reader at 37° C. in the dark, and incubate for 60 minutes. Set the parameters of the microplate reader (run for 30 min, and read a value every 2 min; EX/EM=523/612 nm; Strength: medium to high, sample placement area, shaking). Add 25 μL of hydrogen peroxide solution to 96-well plate, and 25 μL of sodium molybdate solution to 96-well plate. Immediately put it into the microplate reader to run.

Various kinds of plant extracts were screened by Singlet Oxygen Absorption Capacity (SOAC), and it was found that basil extract, oregano extract, cinnamon extract, Sambucus williamsii extract, etc. or combination thereof have higher SOAC value, preferably up to 4,000 μmole TE/g.

### Example 2

The plant-derived composition is prepared from the following components:

| | | | |
|---|---|---|---|
| quercetin | 35%; | kale extract | 6%; |
| olive extract | 48%; | Rosmarinus officinalis extract | 11%. |

### Example 5

The plant-derived composition is prepared from the following components:

| | | | |
|---|---|---|---|
| quercetin | 32%; | kale extract | 5%; |
| olive extract | 50%; | Rosmarinus officinalis extract | 13%. |

### Example 4

The plant-derived composition is prepared from the following components:

| | | | |
|---|---|---|---|
| apple extract | 22%; | blackberry extract | 25%; |
| olive extract | 20%; | resveratrol | 20%; |
| grape seed extract | 13%. | | |

### Example 5

The plant-derived composition is prepared from the following components:

| | | | |
|---|---|---|---|
| green tea extract | 15%; | Acerola cherry extract | 15%; |
| blueberry extract | 10%; | aronia melanocarpa extract | 5%; |
| blackberry extract | 5%; | olive extract | 15%; |
| olive extract | 15%; | resveratrol | 15%; |
| Rosmarinus officinalis extract | 2%; | grape seed extract | 3%. |

### Example 6

One or more of the first plant component including quercetin, green tea extract, broccoli bud extract, onion extract, apple extract, tomato extract, broccoli extract, Camu fruit extract, Acerola cherry extract, blueberry extract, Prunus avium extract, raspberry extract, spinach extract, aronia melanocarpa extract, kale extract, blackberry extract, cowberry extract, was selected, and the content was 30%-50% by weight; one or more of the second plant component including olive extract, olive extract, resveratrol, was selected, and the content was 37%-57% by weight; one or more of the third plant component including Rosmarinus officinalis extract, grape seed extract, was selected, and the content was 5%-20% by weight; and the total weight of the composition is 100%.

### Example 7

The plant-derived composition is prepared from the following components:

| | | | |
|---|---|---|---|
| quercetin | 35%; | kale extract | 5%; |
| olive extract | 47%; | Rosmarinus officinalis extract | 10%; |
| turmeric extract | 1.5%; | cinnamon extract | 1.5%. |

### Example 8

The plant-derived composition is prepared from the following components:

| | | | |
|---|---|---|---|
| quercetin | 40%; | kale extract | 10%; |
| olive extract | 35%; | Rosmarinus officinalis extract | 12%; |
| turmeric extract | 1.3%; | cinnamon extract | 1.3%; |
| resistant dextrin | 0.3%; | silicon dioxide | 0.09%; |
| ergothioneine | 0.01%. | | |

### Example 9

The plant-derived composition is prepared from the following components:

| | | | |
|---|---|---|---|
| Acerola cherry extract | 35%; | raspberry extract | 15%; |
| olive extract | 10%; | olive extract | 8%; |
| resveratrol | 10%; | Rosmarinus officinalis | 9%; |
| | | extract | |
| grape seed extract | 8%; | oregano extract | 1%; |
| cinnamon extract | 1.5%; | basil extract | 0.5%; |
| Sambucus williamsii extract | 1%; | maltodextrin | 0.5%; |
| silicon dioxide | 0.1%; | sodium D-erythorbate | 0.4%. |

### Example 10

The plant-derived composition is prepared from the following components:

| | | | |
|---|---|---|---|
| aronia melanocarpa extract | 25%; | kale extract | 10%; |
| blackberry extract | 5%; | cowberry extract | 5%; |
| olive extract | 23%; | olive extract | 17%; |
| Rosmarinus officinalis extract | 5%; | grape seed extract | 5%; |
| turmeric extract | 1%; | garlic extract | 0.7%; |
| basil extract | 0.8%; | cinnamon extract | 0.5%; |
| lactose | 1%; | magnesium stearate | 0.5%; |
| sodium D-erythorbate | 0.5%. | | |

### Example 11

The plant-derived composition is prepared from the following components:

| | | | |
|---|---|---|---|
| quercetin | 35%; | olive extract | 47%; |
| Rosmarinus officinalis extract | 10%; | kale extract | 5%; |

olive extract, green tea extract, onion extract, Acerola cherry extract, peppermint leaf extract, grape seed extract, curry extract, Sambucus williamsii concentrate, mangosteen concentrate, black currant extract, blueberry extract, cowberry extract, Brussels sprout concentrate, broccoli bud concentrate, white pepper extract, apple extract, tomato concentrate, broccoli concentrate, Camu fruit concentrate, garlic concentrate, oregano concentrate, carrot concentrate, Prunus avium concentrate, raspberry concentrate, Aronia melanocarpa concentrate, blackberry concentrate (0.1% each);

| | | | |
|---|---|---|---|
| resistant dextrin | 0.3%; | silicon dioxide | 0.09%; |
| ergothioneine | 0.01%. | | |

### Example 12

The plant-derived composition is prepared from the following components:

One or more of the first plant component including quercetin, green tea extract, broccoli bud extract, onion extract, apple extract, tomato extract, broccoli extract, Camu fruit extract, Acerola cherry extract, blueberry extract, Prunus avium extract, raspberry extract, spinach extract, aronia melanocarpa extract, kale extract, blackberry extract, cowberry extract, was selected, and the content was 30%-50% by weight; one or more of the second plant component including olive extract, olive extract, resveratrol, was selected, and the content was 37%-57% by weight; one or more of the third plant component including Rosmarinus officinalis extract, grape seed extract, was selected, and the content was 5%-20% by weight; one or more of the fourth plant component including turmeric extract, garlic extract, basil extract, oregano extract, cinnamon extract, carrot extract, Sambucus williamsii extract, mangosteen extract, was selected, and the content was 0.5%-2% by weight; one or more of the fifth plant component including basil extract, oregano extract, cinnamon extract, Sambucus williamsii extract, was selected, and the content was 0.5%-2% by weight; and the total weight of the composition is 100%.

### Example 13 Comparative Example

An existing composite composition on the market:

| | | | |
|---|---|---|---|
| Acerola cherry berry extract | 30%; | quercetin | 25%; |
| Camu fruit berry extract | 15%; | Phyllanthus emblica berry | 15%; |
| | | extract | |
| Acai Berry juice powder | 10%; | wolfberry berry extract | 5%. |

### Example 14 Comparative Example

An existing composite composition on the market:

| | | | |
|---|---|---|---|
| apple fiber (237 mg) | 32.6%; | Acai Berry (215 mg) | 30%; |
| mixed extracts (strawberry, | 20%; | | |
| blueberry, blackberry, raspberry, | | | |
| cranberry, wolfberry) (150 mg) | | | |
| Acerola cherry pulp juice | 15%; | pomegranate juice | 1.4%. |
| powder (110 mg) | | powder (10 mg) | |

### Example 16 Total-ORAC Detection

Total-ORAC is the sum of five indexes: Oxygen Radical Absorbance Capacity (ORAC), Hydroxyl Radical Absorbance Capacity (HORAC), Superoxide anion Radical Absorbance Capacity (SORAC), peroxynitrite [ONOO⁻] Radical Absorbance Capacity (NORAC) and Singlet Oxygen Absorption Capacity (SOAC). The Total-ORAC value was obtained after five kinds of free radical scavenging abilities of different compositions were tested: Oxygen Radical Absorbance Capacity (ORAC), Hydroxyl Radical Absorbance Capacity (HORAC), Superoxide anion Radical Absorbance Capacity (SORAC), peroxynitrite [ONOO⁻] Radical Absorbance Capacity (NORAC) and Singlet Oxygen Absorption Capacity (SOAC). The results are shown in Table 1.

TABLE 1
Test results of Total-ORAC

| | Total-ORAC (μmole Te/g) |
|---|---|
| Example 2 | 21090 |
| Example 3 | 20450 |
| Example 4 | 21652 |
| Example 5 | 22764 |
| Example 7 | 23097 |
| Example 8 | 28365 |
| Example 9 | 28712 |
| Example 10 | 30922 |
| Example 11 | 37048 |
| Example 13 | 14045 |
| (Comparative example) | |
| Example 14 | 15600 |
| (Comparative example) | |

The Total-ORAC values of Example 6 and Example 12 are both greater than 20,000 μmole TE/g, preferably greater than 30,000 μmole TE/g, and more preferably greater than 35,000 μmole TE/g.

### Example 17

The smell of the composition of the invention was compared with that of commercial products. Compared with other products, the smell, taste of the composition of the invention is more acceptable, and the formula product of the invention is not easy to agglomerate or oxidize, which is more conducive to preservation.

The plant-derived composition of the invention is easy to obtain, the source is green and environment-friendly, the ingredients are safe, and it is easy to be absorbed by intestinal tract. It smells and tastes good and improves the adaptability of users. It has high antioxidant capacity, and can efficiently scavenge human free radicals and prevent or inhibit various disorders, diseases or problems caused by excessive free radicals in the human body. Furthermore, the Total-ORAC of the composition of the invention is more than 20,000 μmole TE/g, which is far superior to the existing market products. Preferably, it is greater than 30,000 μmole TE/g, more preferably greater than 35,000 μmole TE/g. As for the composition of the invention, through reasonable formula and proportion, various antioxidant components can produce synergistic effect, and the complementary effect of their structure-activity relationship can play a stronger role than a single antioxidant. Compared with other products on the market, the composition of the invention is more acceptable to consumers in smell and/or taste, and the formula product of the invention is not easy to agglomerate or oxidize.

The above are only the preferred embodiments of the present invention, and are not intended to limit the present invention. Those skilled in the art can make many changes, modifications, substitutions and variations on these embodiments without departing from the principles and purposes of the invention, and the scope of the invention is defined by the claims and their equivalents.

What is claimed is:

1. A plant-derived composition comprises a first plant component, a second plant component and a third plant component, wherein the first plant component is selected from one or more of quercetin, green tea extract, broccoli bud extract, onion extract, apple extract, tomato extract, broccoli extract, Camu fruit extract, Acerola cherry extract, blueberry extract, Prunus avium extract, raspberry extract, spinach extract, Aronia melanocarpa extract, kale extract, blackberry extract, cowberry extract; the second plant component is selected from one or more of olive extract, clove extract, resveratrol; the third plant component is selected from one or more of Rosmarinus officinalis extract, grape seed extract.

2. The plant-derived composition of claim 1, wherein the plant-derived composition comprises, by weight, 30%-50% of the first plant component, 37%-57% of the second plant component and 5%-20% of the third plant component.

3. The plant-derived composition of claim 1, wherein the plant-derived composition further comprises 0.5%-2% by weight of a fourth plant component, and the fourth plant component is selected from one or more of turmeric extract, garlic extract, basil extract, oregano extract, cinnamon extract, carrot extract, Sambucus williamsii extract, mangosteen extract.

4. The plant-derived composition of claim 1, wherein the plant-derived composition further comprises 0.5%-2% by weight of a fifth plant component, and the fifth plant component is selected from one or more of basil extract, oregano extract, cinnamon extract, Sambucus williamsii extract.

5. The plant-derived composition of claim 1, wherein the plant-derived composition further comprises a filler, a glidant, or an additive.

6. The plant-derived composition of claim 5, wherein the filler includes one or more of resistant dextrin, maltodextrin, lactose; the glidant includes one or more of silicon dioxide, magnesium stearate, talcum powder, tricalcium phosphate; the additive includes one or more of ergothioneine, L-ascorbic acid, sodium D-erythorbate, tea polyphenol.

7. The plant-derived composition of claim 1, wherein the first plant component is quercetin, kale extract; the second plant component is olive extract; the third plant component is Rosmarinus officinalis extract.

8. The plant-derived composition of claim 3, wherein the fourth plant component is turmeric extract;

9. The plant-derived composition of claim 4, wherein the fifth plant component is cinnamon extract.

10. The plant-derived composition of claim 1, wherein the plant-derived composition further comprises resistant dextrin, silicon dioxide, ergothioneine.

11. The plant-derived composition of claim 1, wherein the plant-derived composition is prepared in powder form.

12. The plant-derived composition of claim 1, wherein the plant-derived composition is administered once or more times a day.

13. The plant-derived composition of claim 1, wherein the plant-derived composition is prepared as foods, beverages, nutraceuticals, dietary supplements, cosmetics.
